(19) United States
(12) Patent Application Publication
(10) Pub. No.: US 2026/0216466 A1
(43) Pub. Date: Jul. 30, 2026

(54) OXYGEN HUMIDIFIER AND METHODS OF USE

(71) Applicant: Flo Ez LLC, Logan, UT (US)

(72) Inventors: Zachary E. Humes, North Logan, UT (US); Nathan D. McCulloch, Holiday, UT (US); James Robbins, Logan, UT (US); Nathan Erickson, Beaver Dam, UT (US); Joseph Foley, Denver, CO (US); Spencer Johnson, Orem, UT (US); Jonathan Clark, JR., Hyde Park, UT (US); David K. Webb, Smithfield, UT (US)

(21) Appl. No.: 19/465,964

(22) Filed: Jan. 30, 2026

Related U.S. Application Data
(60) Provisional application No. 63/751,724, filed on Jan. 30, 2025.

(51) Int. Cl. A61M 16/16; A61M 16/20

(52) U.S. Cl. CPC A61M 16/16; A61M 16/209; A61M 2202/0208

(57) ABSTRACT

Disclosed herein is an oxygen humidifier that includes a container defining an interior space and an opening, a lid coupled to the container, and at least one latch system configured to releasably secure the lid to the container. The lid includes an inlet port and an outlet port fixed to the lid. The lid is movable between an open position, in which the lid is unsealed from the container to provide access to the interior space, and a closed position, in which the lid forms a seal with the container. In some examples, the lid is pivotably coupled to the container by a hinge. In other examples, a multiple latch systems enables removal of the lid from the container by non-rotational separation. The humidifier may further include a diffuser positioned within the interior space and a pressure relief valve coupled to the lid.

### CROSS-REFERENCE TO RELATED APPLICATION

This application claims the benefit of U.S. Provisional Patent Application No. 63/751,724, filed January 30, 2025, which is incorporated herein by reference in its entirety.

### FIELD

This disclosure relates generally to oxygen humidifiers, and more particularly to oxygen humidifiers for supplying humidified oxygen in medical applications.

### BACKGROUND

Oxygen humidifiers are commonly used to humidify dry oxygen prior to inhalation, particularly for individuals with sensitive lungs requiring assisted breathing. Such devices typically channel dry oxygen from an oxygen source into a water-filled container through an inlet port. The oxygen is released into the water, allowing moisture to be entrained in the oxygen as it passes through the container. The resulting humidified oxygen is then supplied to a user through an outlet line connected to a breathing apparatus.

Many oxygen humidifiers include a container and a lid that is removable to allow refilling of water depleted during use. In some conventional designs, the lid is secured to the container using threaded or rotational coupling, which requires the lid to be rotated relative to the container during opening and closing. In practice, this can increase the likelihood of misalignment during reattachment and may require inlet and outlet tubing to be disconnected, repositioned, or managed during opening and closing of the lid to avoid entanglement. In addition, some conventional designs may exhibit reduced efficiency in oxygen distribution within the water, particularly at relatively low oxygen flow rates, due to releasing oxygen for a limited number of outlets or in a single direction, which can reduce dispersion within the water.

### SUMMARY

The subject matter of the present application has been developed in response to the present state of the art, and particularly in response to the shortcomings associated with conventional oxygen humidifier approaches that have not yet been fully solved by currently available techniques. Accordingly, the subject matter of the present application has been developed to provide an oxygen humidifier, that overcome at least some of the above-mentioned shortcomings of prior art techniques.

The following is a non-exhaustive list of examples, which may or may not be claimed, of the subject matter, disclosed herein.

Disclosed herein is an oxygen humidifier including a container defining an interior space and an opening for accessing the interior space. The oxygen humidifier also includes a lid pivotably coupled to the container via a hinge that defines a rotation axis about which the lid pivots relative to the container. The oxygen humidifier further includes a latch system including a latch coupled to the lid and a latch receiver coupled to the container. The latch and latch receiver are configured to releasably secure the lid to the container. The lid includes an inlet port and an outlet port fixed to the lid. The lid is selectively pivotable about the rotation axis between an open position, in which the lid is unsealed against the container to enable access to the interior space through the opening of the container, and a closed position, in which the lid forms a seal with the container. When the lid pivots from the closed position to the open position, the lid pivots away from and remains outside of the interior space. The preceding subject matter of this paragraph characterizes example 1 of the present disclosure.

The latch of the latch system defines a latch opening. The latch receiver of the latch system includes a release button and a locking projection operatively coupled to the release button. The locking projection is configured to engage the latch opening to retain the lid in the closed position, and actuation of the release button disengages the locking projection from the latch opening. The preceding subject matter of this paragraph characterizes example 2 of the present disclosure, wherein example 2 also includes the subject matter according to example 1, above.

The latch system is positioned on the lid at a location opposite the hinge relative to the opening of the container. The preceding subject matter of this paragraph characterizes example 3 of the present disclosure, wherein example 3 also includes the subject matter according to example 1 or 2, above.

The oxygen humidifier further includes a diffuser fluidically coupled to the inlet port and positioned within the interior space of the container when the lid is in the closed position. The diffuser includes a plurality of lateral passages formed in a sidewall of the diffuser and oriented to expel a fluid in a direction substantially normal to a longitudinal axis of the diffuser, and at least one axial passage formed in a distal end of the diffuser and oriented to expel the fluid in a direction substantially parallel to the longitudinal axis of the diffuser. The preceding subject matter of this paragraph characterizes example 4 of the present disclosure, wherein example 4 also includes the subject matter according to any of examples 1-3, above.

The at least one axial passage has a tapered cross-section in a direction away from the lid. The preceding subject matter of this paragraph characterizes example 5 of the present disclosure, wherein example 5 also includes the subject matter according to example 4, above.

The oxygen humidifier further includes a pressure relief valve coupled to the lid. The pressure relief valve includes a housing defining an interior channel and at least one exhaust port in fluid communication with the interior channel. The housing has a transparent portion and a valve member positioned within the interior channel and movable between a closed position, in which the valve member covers the at least one exhaust port, and an open position, in which the valve member uncovers the at least one exhaust port. The transparent portion enables observation of the valve member when the pressure relief valve is in the open position. The preceding subject matter of this paragraph characterizes example 6 of the present disclosure, wherein example 6 also includes the subject matter according to any of examples 1-5, above.

The oxygen humidifier further includes a gasket coupled within a recessed groove of an interior surface of the lid. The gasket is configured to sealingly engage a rim of the container that defines the opening when the lid is in the closed position to form a seal between the lid and the container, and the recessed groove is defined between opposing wall portions of the interior surface that retain the gasket. The preceding subject matter of this paragraph characterizes example 7 of the present disclosure, wherein example 7 also includes the subject matter according to any of examples 1-6, above.

The container has at least one planar portion on an exterior surface of the container. The preceding subject matter of this paragraph characterizes example 8 of the present disclosure, wherein example 8 also includes the subject matter according to any of examples 1-7, above.

The oxygen humidifier further includes one or more protective ribs formed on the container adjacent to the latch receiver, the one or more protective ribs being positioned to shield the latch receiver from inadvertent actuation. The preceding subject matter of this paragraph characterizes example 9 of the present disclosure, wherein example 9 also includes the subject matter according to any of examples 1-8, above.

The inlet port includes internal threads formed on an inner surface of the inlet port. The internal threads are configured to engage a corresponding threaded connector of an inflow tube. The preceding subject matter of this paragraph characterizes example 10 of the present disclosure, wherein example 10 also includes the subject matter according to any of examples 1-9, above.

Engagement between the latch and the latch receiver provides an audible indication that the lid is secured in the closed position. The preceding subject matter of this paragraph characterizes example 11 of the present disclosure, wherein example 11 also includes the subject matter according to any of examples 1-10, above.

Further disclosed herein is an oxygen humidifier including a container defining an interior space and an opening for accessing the interior space, a lid removably coupled to the container and a multiple latch systems. Each one of the multiple latch systems includes a latch coupled to the lid and a latch receiver coupled to the container. The latch and latch receiver are configured to releasably secure the lid to the container. The lid includes an inlet port and an outlet port fixed to the lid. The lid is selectively movable between an open position, in which the lid is unsealed against the container to enable access to the interior space through the opening of the container, and a closed position, in which the lid forms a seal with the container. When the lid is in the closed position, the latch and the latch receiver of each one of the multiple latch systems are engaged to retain the lid in sealed engagement with the container. When the lid is moved from the closed position to the open position, actuation of the latch receiver of each one of the multiple latch systems disengages the corresponding latch to enable separation of the lid from the container without rotation of the lid relative to the container about a central axis of the container. The preceding subject matter of this paragraph characterizes example 12 of the present disclosure.

The multiple latch systems includes two latch systems including a first latch system and a second latch system. The first latch system is positioned opposite of the second latch system on the lid relative to the opening of the container. The preceding subject matter of this paragraph characterizes example 13 of the present disclosure, wherein example 13 also includes the subject matter according to example 12, above.

The latch of each one of the multiple latch systems defines a latch opening. The latch receiver of each one of the multiple latch systems includes a release button and a locking projection operatively coupled to the release button. The locking projection is configured to engage the latch opening to retain the lid in the closed position, and actuation of the release button disengages the locking projection from the latch opening. The preceding subject matter of this paragraph characterizes example 14 of the present disclosure, wherein example 14 also includes the subject matter according to example 12 or 13, above.

One or more protective ribs are formed on the container adjacent to the latch receiver of each one of the multiple latch systems. The one or more protective ribs are positioned to shield the latch receiver from inadvertent actuation. The preceding subject matter of this paragraph characterizes example 15 of the present disclosure, wherein example 15 also includes the subject matter according to any of examples 12-14, above.

Engagement between the latch and the latch receiver of each one of the multiple latch systems provides an audible indication that the corresponding one of the multiple latch systems is secured. The preceding subject matter of this paragraph characterizes example 16 of the present disclosure, wherein example 16 also includes the subject matter according to any of examples 12-15, above.

Further disclosed herein is a method of accessing an interior space of an oxygen humidifier. The method includes actuating a latch receiver of at least one latch system to disengage a latch from the latch receiver. The latch receiver is coupled to a container of the oxygen humidifier and the latch being coupled to the lid, thereby unsealing the lid from the container. The method also includes moving the lid relative to the container from a closed position to an open position without rotating the lid relative to the container about a central axis of the container, while maintaining fluid tubing connected to an inlet port and an outlet port of the lid. The preceding subject matter of this paragraph characterizes example 17 of the present disclosure.

In some examples, moving the lid relative to the container includes pivoting the lid about a hinge while the lid remains attached to the container. The preceding subject matter of this paragraph characterizes example 18 of the present disclosure, wherein example 18 also includes the subject matter according to example 17, above.

In other examples, moving the lid relative to the container comprises separating the lid from the container by linear separation after disengaging a multiple latch systems. The preceding subject matter of this paragraph characterizes example 19 of the present disclosure, wherein example 19 also includes the subject matter according to example 17, above.

The method further includes moving the lid relative to the container from the open position to the closed position without rotating the lid relative to the container, and receiving an audible indication upon engagement of the latch and the latch receiver of the at least one latch system when the lid is in the closed position. The preceding subject matter of this paragraph characterizes example 20 of the present disclosure, wherein example 20 also includes the subject matter according to any of examples 17-19, above.

The described features, structures, advantages, and/or characteristics of the subject matter of the present disclosure may be combined in any suitable manner in one or more examples and/or implementations. In the following description, numerous specific details are provided to impart a thorough understanding of examples of the subject matter of the present disclosure. One skilled in the relevant art will recognize that the subject matter of the present disclosure may be practiced without one or more of the specific features, details, components, materials, and/or methods of a particular example or implementation. In other instances, additional features and advantages may be recognized in certain examples and/or implementations that may not be present in all examples or implementations. Further, in some instances, well-known structures, materials, or operations are not shown or described in detail to avoid obscuring aspects of the subject matter of the present disclosure. The features and advantages of the subject matter of the present disclosure will become more fully apparent from the following description and appended claims, or may be learned by the practice of the subject matter as set forth hereinafter.

### BRIEF DESCRIPTION OF THE DRAWINGS

In order that the advantages of the subject matter may be more readily understood, a more particular description of the subject matter briefly described above will be rendered by reference to specific examples that are illustrated in the appended drawings. Understanding that these drawings, which are not necessarily drawn to scale, depict only certain examples of the subject matter and are not therefore to be considered to be limiting of its scope, the subject matter will be described and explained with additional specificity and detail through the use of the drawings, in which:

FIG. 1A is a cross-sectional side elevation view of one example of an oxygen humidifier having a container and lid coupled to the container, taken along a plane similar to the plane 1A-1A of FIG. 5A, according to one or more examples of the present disclosure;

FIG. 1B is a cross-sectional side elevation view of the oxygen humidifier of FIG. 1A taken along a plane similar to the plane 1A-1A and further illustrating a liquid within the container and inflow and outflow tubes connected to the lid, according to one or more examples of the present disclosure;

FIG. 2 is a perspective side view of one example of an oxygen humidifier, illustrating the lid in a closed position relative to the container, according to one or more examples of the present disclosure;

FIG. 3 is a perspective side view of the oxygen humidifier of FIG. 2, illustrating the lid in an open position relative to the container, according to one or more examples of the present disclosure;

FIG. 4 is a perspective side view of another example of an oxygen humidifier having multiple latch systems, according to one or more examples of the present disclosure;

FIG. 5A is a top view of an exterior surface of a lid of one example of an oxygen humidifier, according to one or more examples of the present disclosure;

FIG. 5B is a top view of an interior surface of the lid of FIG. 5A, according to one or more examples of the present disclosure;

FIG. 6A is a perspective transparent view of one example of a diffuser for an oxygen humidifier, according to one or more examples of the present disclosure;

FIG. 6B is a bottom view of the diffuser of FIG. 6A, according to one or more examples of the present disclosure;

FIG. 6C is a cross-sectional side view of the diffuser of FIG. 6B, taken along plane B-B of FIG. 6B, illustrating axial passages within the diffuser, according to one or more examples of the present disclosure;

FIG. 7A is a cross-sectional view of the oxygen humidifier of FIG. 1A, taken along a plane similar to the plane 1A-1A of FIG. 5A and illustrating a valve member of a pressure relief valve in a closed position, according to one or more examples of the present disclosure;

FIG. 7B is a cross-sectional side view of the oxygen humidifier of FIG. 1A, taken along a plane similar to the plane 1A-1A of FIG. 5A and illustrating the valve member of the pressure relief valve in an open position, according to one or more examples of the present disclosure; and

FIG. 8 is a schematic flow diagram of a method of accessing an interior space of an oxygen humidifier, according to one or more examples of the present disclosure.

### DETAILED DESCRIPTION

Reference throughout this specification to “one example,” “an example,” or similar language means that a particular feature, structure, or characteristic described in connection with the example is included in at least one example of the present disclosure. Appearances of the phrases “in one example,” “in an example,” and similar language throughout this specification may, but do not necessarily, all refer to the same example. Similarly, the use of the term “implementation” means an implementation having a particular feature, structure, or characteristic described in connection with one or more examples of the present disclosure, however, absent an express correlation to indicate otherwise, an implementation may be associated with one or more examples.

Disclosed herein is an oxygen humidifier for humidifying a flow of fluid for use in breathing-assistance applications. The oxygen humidifier includes a container for holding a liquid and a lid coupled to the container. The lid includes an inlet port for directing a gas into the container and an outlet port for delivering humidified gas to a user (e.g., patient). The inlet port and the outlet port are fixed to the lid such that the inlet port and the outlet port move with the lid as the lid is opened and closed.

The lid is configured to be opened and closed relative to the container without rotating the lid relative to the container about a central axis of the container, which enables the lid to be opened without disconnecting or risk entanglement of tubing coupled to the inlet port or the outlet port. Accordingly, the lid is secured to the container using one or more latches that retain the lid in a sealed condition when closed. Unlike oxygen humidifiers that rely on threaded or rotational lid engagement, this non-rotational opening configuration facilitate refilling and maintenance of the container while reducing the need to manage inlet and outlet tubing during opening and closing of the lid.

In some examples, the lid is pivotably coupled to the container via a hinge such that the lid remains coupled to the container as the lid moves between an open position and a closed position. In such examples, at least one latch cooperates with the hinge to retain the lid in the closed position and maintain a sealed condition between the lid and the container. The pivotable coupling enables the lid to move away from the container opening during opening without entering the interior space of the container. In other examples, the lid is selectively removable from the container (via a hingeless connection) and is secured to the container using multiple latches. The latches are configured to releasably secure the lid to the container in the closed position without requiring threaded or rotational engagement between the lid and the container. When the latches are released, the lid may be removed to allow access to the interior space of the container for refilling or maintenance. The oxygen humidifier may also include a diffuser positioned within the container for distributing oxygen through the water, a pressure relief valve configured to provide audible and visual indications of pressure conditions within the container, and a gasket positioned between the lid and the container to form an airtight seal.

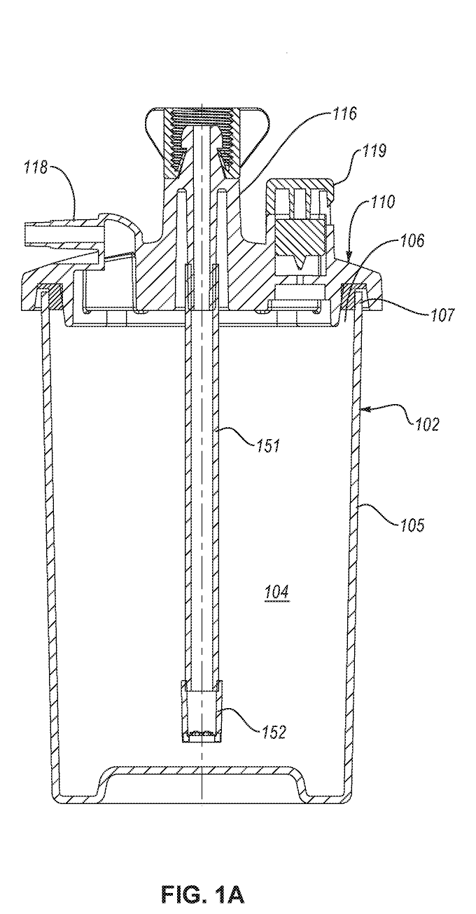

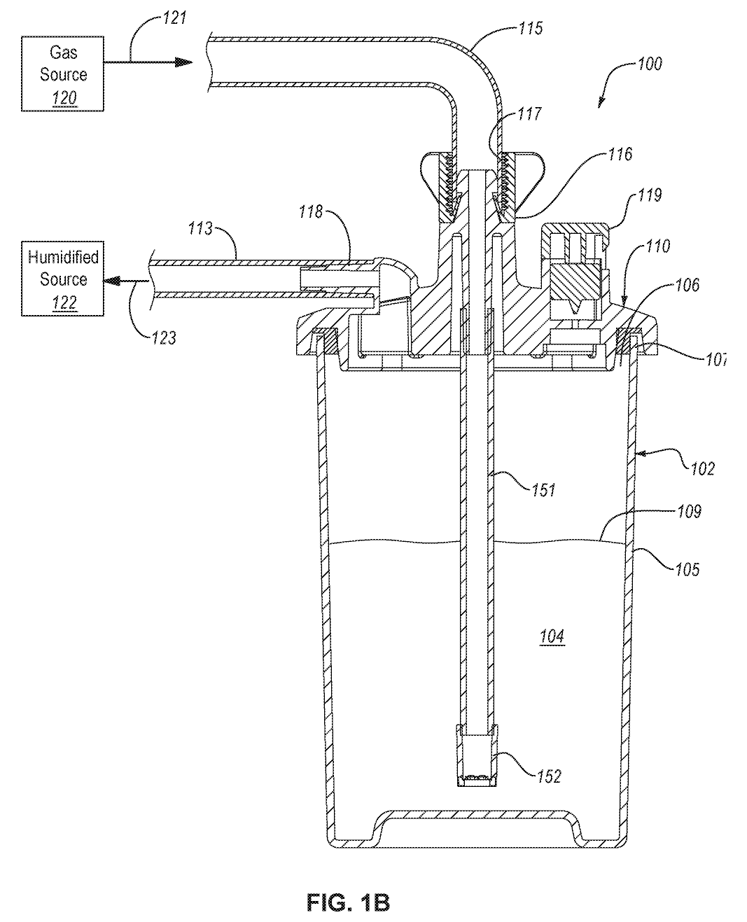

Referring to FIGS. 1A and 1B, one example of an oxygen humidifier 100 (hereinafter “humidifier”) includes a container 102 and a lid 110. As shown in FIG. 1A, the container 102 defines an interior space 104 for holding a liquid 109 (e.g., water) and includes an opening 106 at an upper end of the container 102 that provides access to the interior space 104. The lid 110 is configured to cover the opening 106 of the container 102 and includes an inlet port 116 and an outlet port 118 fixed to the lid 110. As shown in FIG. 1B, the inlet port 116 and the outlet port 118 of the lid 110 may be coupled to respective inflow and outflow tubes that transport a gas into and out of the humidifier 100, respectively. As used herein, the term gas refers to a compressible fluid capable of flowing, such as oxygen or other respiratory gases. For convenience, the humidifier 100 is described with reference to oxygen; however, it is understood that other gases may also be used with the humidifier 100.

The humidifier 100 includes a fluid conduit 151 positioned within the container 102 and extending from the inlet port 116 to a diffuser 152. The fluid conduit 151 is fluidically coupled to the inlet port 116 and configured to convey a gas from the inlet port 116 into the interior space 104 of the container 102 via the diffuser 152, such that a gas delivered through the inlet port 116 is directed into the liquid 109 contained within the container 102. In some examples, the fluid conduit 151 and the diffuser 152 are centered within the interior space 104 or aligned with a central axis (C) of the container 102. In other examples, the fluid conduit 151 and the diffuser 152 are offset relative to the central axis (C) of the container 102.

In some examples, the humidifier 100 is configured to humidify a respiratory gas for delivery to a patient. For example, the respiratory gas may include oxygen, which may be humidified by passing the oxygen through the liquid 109 prior to delivery to the patient. In certain examples, the humidifier 100 may be used in high-flow oxygen applications, such as delivery through a mask or a nasal cannula.

As shown in FIG. 1B, the humidifier 100 humidifies a flow of gas entering the humidifier 100 via the inlet port 116 (see, e.g., directional arrow 121) to produce humidified gas 122, which exits the humidifier 100 via the outlet port 118 (see, e.g., directional arrow 123). An inflow tube 115 coupled to the inlet port 116 is in fluid-receiving communication with a gas source 120. The gas source 120 may include, for example, an oxygen tank, an oxygen concentrator, or another medical-grade oxygen supply system capable of delivering a controlled flow of oxygen. An outflow tube 113 couples the outlet port 118 to a gas delivery device, such as a nasal cannula, through which the humidified gas 122 is delivered to the patient. The fluid conduit 151 directs the gas from the inlet port 116 to the diffuser 152, which is positioned within the container 102 at a location configured to be submerged beneath a minimum operating amount of the liquid 109 during use. In one example, the liquid 109 is purified water.

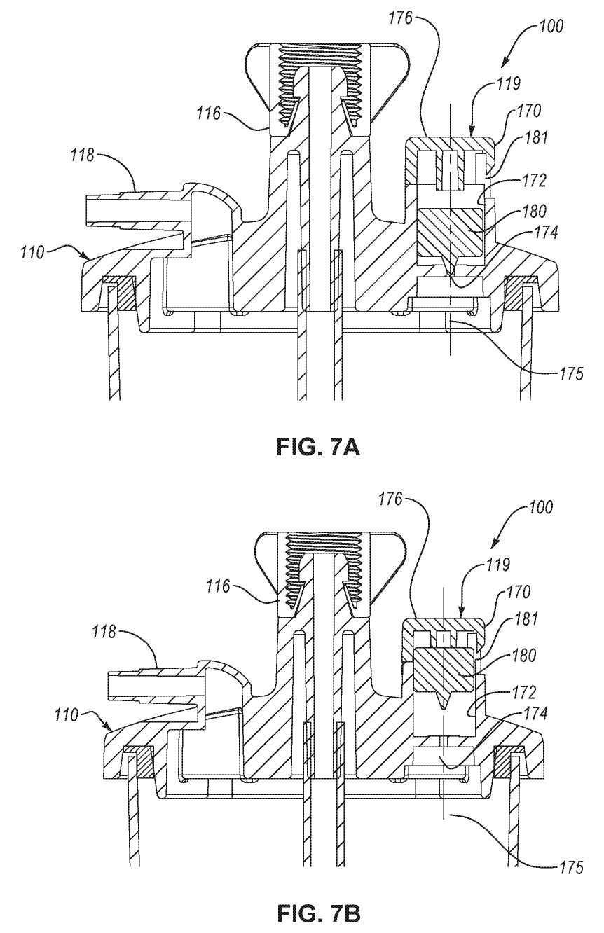

In some examples, the humidifier 100 further includes a pressure relief valve 119 coupled to the lid 110 and configured to relieve excess pressure within the container 102. The pressure relief valve 119 may be configured to vent gas from the container 102 when a pressure condition exceeding a predetermined level is present within the container 102, thereby reducing the likelihood of over-pressurization during use. In some examples, the pressure relief valve 119 is further configured to provide an indication of a pressure relief event. The structure and operation of the pressure relief valve 119 will be described in greater detail below with reference to FIGS. 7A and 7B.

The container 102 may be formed partially or entirely from a rigid polymeric material, such as ABS, polyurethane, polycarbonate, PET, or other suitable materials. In some examples, the container 102 is transparent, enabling a user to observe the fluid conduit 151, the diffuser 152, and/or a level of the liquid 109 within the interior space 104. In other examples, the container 102 may be opaque. When transparent materials are used, the container 102 may include markings on an exterior surface 105 that indicate one or more recommended fill levels for the liquid 109 (e.g., maximum or minimum).

In some examples, the container 102 has a generally circular cross-sectional shape. In other examples, the container 102 has a non-circular cross-sectional shape. For example, the container 102 may include at least one planar portion formed on the exterior surface 105 of the container 102, with the planar portion extending along at least a portion of a length of the container 102. The planar portion may facilitate handling of the container 102, such as by providing an improved gripping surface during filling or transport, and may also provide a stable surface for coupling components to the container 102.

The opening 106 of the container 102 is sized to facilitate refilling of the container 102 with the liquid 109 and to allow insertion and removal of internal components positioned within the interior space 104, such as the fluid conduit 151 and the diffuser 152. The lid 110 is sized to cover an entirety of the opening 106 of the container 102 and is configured to form a sealed interface with the container 102 when in a closed position. In some examples, an outer periphery of the lid 110 extends beyond a rim 107 of the opening 106 such that the lid 110 overlaps the rim 107. In other examples, the outer periphery of the lid 110 is substantially aligned with the rim 107 of the opening 106. The lid 110 may be formed from a rigid polymeric material similar to that of the container 102.

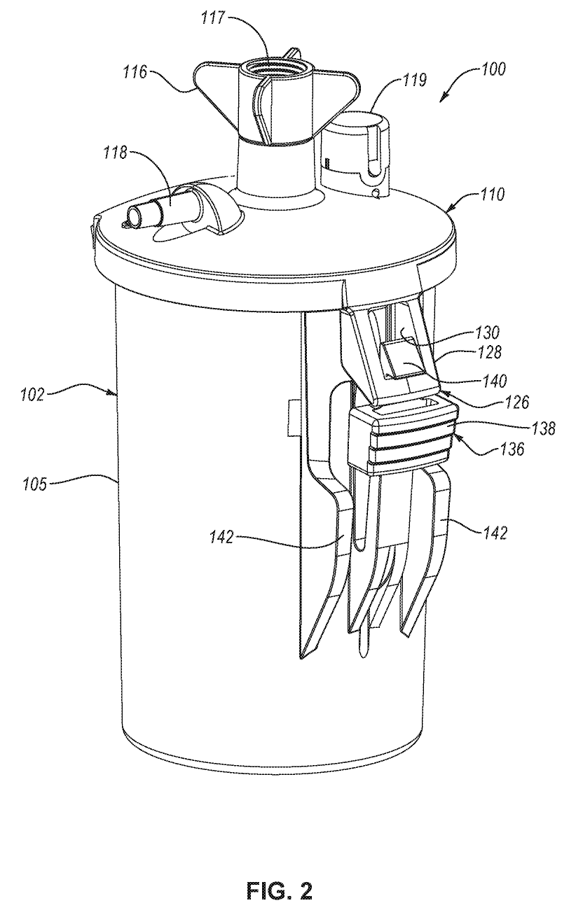

As shown in FIG. 2A, the humidifier 100 includes at least one latch system, which includes a latch 128 coupled to the lid 110 and a latch-receiver 136 coupled to the container 102, where the latch 128 and the latch receiver 136 cooperate to releasably secure the lid 110 relative to the container 102.

The latch 128 is coupled to the lid 110 and moves with the lid 110 as the lid 110 is moved between an open position, in which an entirety of the lid 110 can be unsealed from the container 102 to permit access to the interior space 104, and a closed position, in which an entirety of the lid 110 can be sealed against the container 102 to close the opening 106. As shown, the latch 128 extends from the lid 110 toward the container 102 and includes a latch opening 130 formed therethrough. The latch opening 130 is defined by material of the latch 128 surrounding the opening and is configured to receive a corresponding locking projection of the latch receiver 136 when the lid 110 is in the closed position. That is, the latch opening 130 extends through a thickness of the latch 128 to enable interlocking engagement with the latch receiver 136. The latch 128 may be formed integrally with the lid 110 or may be a separate component that is fixedly attached to the lid 110 using molding, mechanical fastening, or other suitable attachment techniques. In some examples, the latch 128 is formed from a material that permits limited elastic deformation during engagement with the latch receiver 136 while remaining fixed relative to the lid 110. Although a single latch 128 is shown, the humidifier 100 may include multiple latches positioned around the lid 110 to cooperate with corresponding latch receivers 136 distributed around the container 102.

In some examples, the latch 128 is structurally supported from a region below an upper surface of the lid 110. For example, the latch 128 may extend from a lower portion of the lid 110 such that the latch 128 is supported from below rather than being cantilevered from an upper surface of the lid 110. This configuration may increase stiffness of the latch 128 under internal pressure conditions within the container 102 and reduces deflection of the latch 128 when the humidifier 100 is pressurized.

As shown, the latch 128 extends generally downward from the lid 110 when the lid 110 is in the closed position. The latch 128 has a thickness and a cross-sectional profile selected to provide sufficient structural stiffness to resist deformation under internal pressure conditions within the container 102. In some examples, the latch 128 is formed from a polymeric material having sufficient rigidity to maintain engagement with the latch receiver 136 during use, while optionally permitting limited elastic deformation during engagement and disengagement.

The latch receiver 136 is coupled to the exterior surface 105 of the container 102 and is positioned to align with the latch 128 when the lid 110 is in the closed position. The latch receiver 136 may be integrally formed with the container 102 or formed as a separate component that is attached to the container 102. In some examples, the latch receiver 136 includes a movable release button 138 that is accessible to a user and a locking projection 140, operatively coupled to and co-movable with the release button 138. The release button 138 is configured to move inwardly relative to the container 102 in response to user-applied force. The locking projection 140 is configured to selectively engage the latch opening 130 of the latch 128 to retain the lid 110 in the closed position. Actuation of the release button 138 causes corresponding movement of the locking projection 140 between an engaged position, in which the locking projection 140 is received within the latch opening 130 to retain the lid 110 relative to the container 102, and a disengaged position, in which the locking projection 140 is withdrawn from the latch opening 130 to permit release of the lid 110 from the container 102.

In use, when the lid 110 is moved from the open position toward the closed position, the latch 128 is guided into the latch receiver 136. Continued movement of the lid 110 causes the locking projection 140 of the latch receiver 136 to engage the latch opening 130, thereby securing the lid 110 relative to the container 102 and retaining the lid 110 in sealed engagement with the container 102. In some examples, engagement occurs automatically as the lid 110 reaches the closed position. To open or remove the lid 110, a user actuates the release button 138 of the latch receiver 136, which causes the locking projection 140 to move out of engagement with the latch opening 130. Disengagement of the locking projection 140 enables the latch 128 to disengage from the latch receiver 136, thereby allowing the lid 110 to move from the closed position to the open position or to be removed from the container 102.

In some examples, movement of the locking projection 140 relative to the latch opening 130 may be biased toward engagement by a biasing element, such as a spring. In other examples, engagement and disengagement of the locking projection 140 may be achieved through elastic deformation and geometric interaction between components of the latch receiver 136 and the latch 128, without the use of a separate biasing element. For example, the release button 138 may be formed from a resiliently deformable material or include a flexible region that enables temporary deformation during actuation and returns toward an original position when released. The latch system may therefore be implemented using either biased or snap-fit style engagement mechanisms.

In some examples, engagement between the latch 128 and the latch receiver 136 provides a tactile, audible, or visual indication that the lid 110 is properly secured in the closed position. For example, engagement of the locking projection 140 with the latch opening 130 may produce an audible sound, such as a click, that indicates successful engagement of the latch system. In some examples, the audible indication comprises a distinct clicking sound produced upon engagement of the latch 128 with the latch receiver 136. In this manner, the latch system may function as a “go / no-go” indicator to provide user feedback confirming that the lid 110 is fully secured prior to use. Additionally, or alternatively, in some examples, if the latch 128 does not engage properly when the lid 110 is brought to the closed position, the lid 110 may spring open, providing a visual indicator to a user that the humidifier 100 is not properly sealed.

In some examples, the container 102 includes one or more protective ribs 142 positioned adjacent to the latch receiver 136. The protective ribs 142 extend outward from the exterior surface 105 of the container 102 and at least partially surround the latch receiver 136, thereby at least partially shielding the latch receiver 136 from inadvertent actuation. That is, the protective ribs 142 are configured to reduce the likelihood of accidental actuation of the latch system during handling, transport, or incidental contact. In other examples, the protective ribs 142 may be implemented as, or supplemented by, other protective structures, such as flanges, walls, or recessed regions formed in the container 102, that restrict access to the latch receiver 136 except when intentionally actuated by a user.

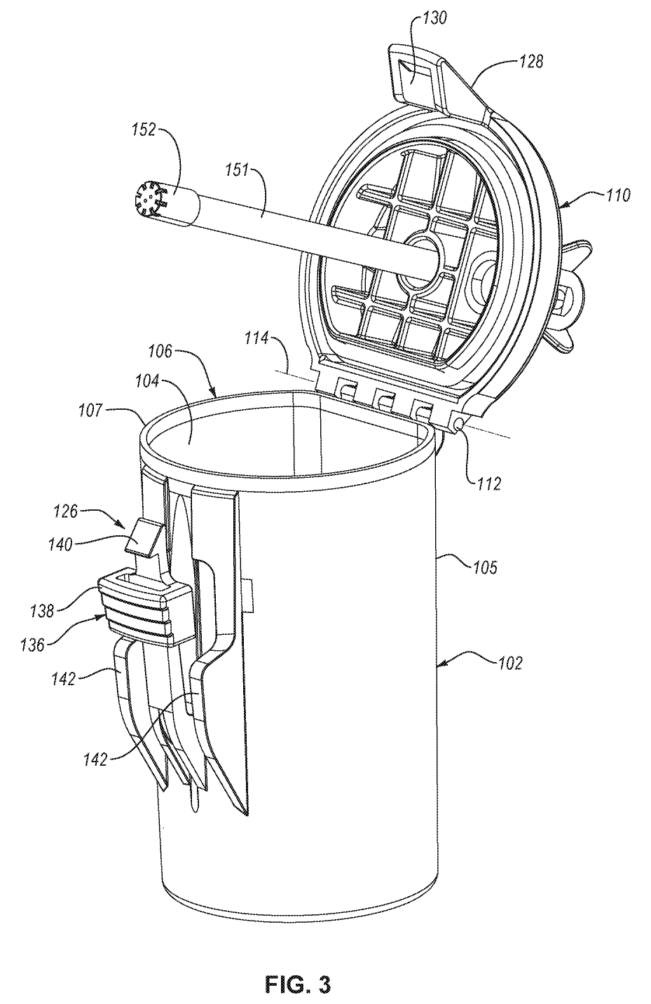

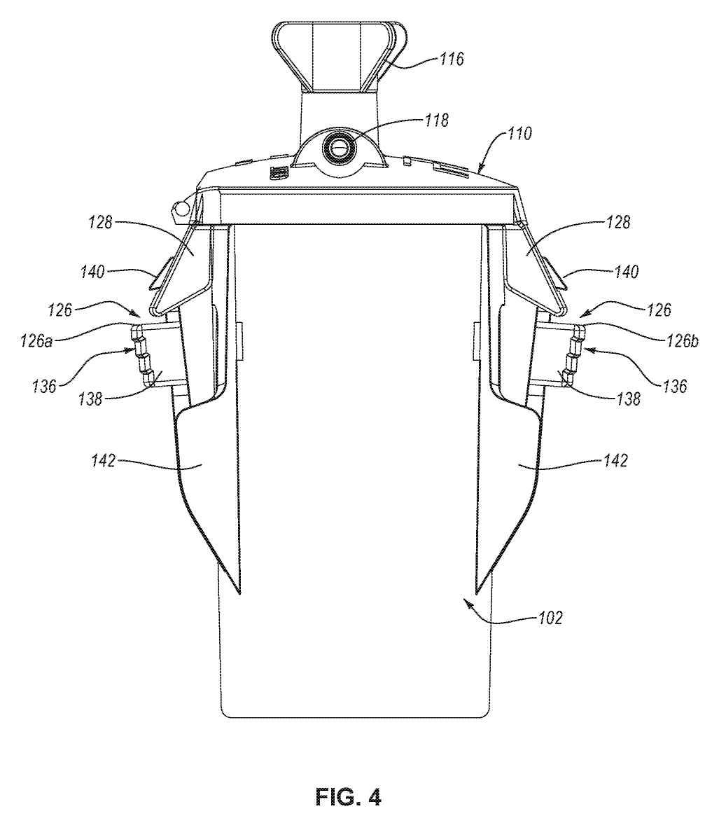

In some examples, the lid 110 is coupled to the container 102 by a hinge in addition to the latch system 126 (i.e., a hinge-and-latch configuration), such that the lid 110 remains attached to the container 102 as the lid 110 is moved between the open position and the closed position (see, e.g., FIG. 3). In other examples, the lid 110 may be coupled to the container 102 using multiple latch systems 126 without a hinge (i.e., a multi-latch configuration), such that release of the multiple latch systems allows the lid 110 to be separated and removed from the container 102 (see, e.g., FIG. 4). The hinge-and-latch configuration and multi-latch configurations provide alternative mechanisms for securing the lid 110 relative to the container 102.

Referring to FIG. 3, in some examples, the humidifier 100 includes a hinge 112 that pivotably couples the lid 110 to the container 102. The hinge 112 defines a rotation axis 114 about which the lid 110 pivots relative to the container 102. Accordingly, movement of the lid 110 in any latch-and-hinge configurations includes angular motion about the rotation axis 114, while remaining attached to the container 102. In this configuration, the lid 110 is permanently attached to the container 102 via the hinge 112 and is not removable during normal opening and closing. When the lid 110 is in the closed position (see, e.g., FIG. 2), at least one latch system retains the lid 110 in sealed engagement with the container 102. When at least one latch system is released, the hinge 112 allows the lid 110 to pivot away from the opening 106 of the container 102 and remain outside of the interior space 104, to provide access to the interior space 104, as shown in FIG. 3 in the open position. In some examples, the latch-and-hinge configuration includes a single latch system, such as a latch system that is opposite of the hinge. In other examples, the latch-and-hinge configuration includes multiple latch systems, such as a latch systems distributed around the lid perimeter to provide additional retention.

Unlike humidifiers that rely on threaded or rotational lid engagement where the lid must be rotated about a central axis of the container to disengage mating threads between the lid and the container, the hinge-and-latch configuration enables the lid 110 to be opened without rotation about the central axis (C) of the container 102 (see, e.g., FIG. 1A). In other words, the lid 110 is not required to rotate about the central axis (C) of the container 102 to transition between the open and closed position, even though the lid 110 may pivot about the rotation axis 114. This enables inlet and outlet tubing coupled to the lid 110 to remain connected during opening and closing, which can reduce the likelihood of tubing entanglement, disconnection, or misalignment during refilling or maintenance.

The hinge 112 may be implemented using any of various hinge configurations suitable for pivotally coupling the lid 110 to the container 102. For example, the hinge 112 may include one or more hinge elements formed on or coupled to the container 102 and corresponding hinge elements formed on or coupled to the lid 110, with a hinge pin extending through aligned apertures to define the rotation axis 114. In some examples, the container 102 has at least one planar portion and the hinge 112 may be positioned along the planar portion of the container 102. The planar portion can provide a stable mounting surface for the hinge 112, facilitate alignment between the lid 110 and the container 102 during opening and closing, improve resistance to rotational or lateral loads applied to the hinge during use, and/or provide an exterior surface that is easier to grip or stabilize during handling of the container 102. In other examples, the hinge 112 may be positioned along a curved portion of the container 102.

Referring to FIG. 4, in some examples the lid 110 of a humidifier 200 is secured to the container 102 using multiple latch systems 126 without a hinge. In this multi-latch configuration, the lid 110 is removably coupled to the container 102 by engagement of the multiple latches 128 with corresponding latch receivers 136 positioned around the container 102. When the multiple latch systems 126 are engaged, the lid 110 is retained in sealed engagement with the container 102. When the multiple latch systems 126 are released, the lid 110 may be lifted away from the container 102 to provide access to the interior space 104.

As shown, the humidifier 200 includes two latch systems 126 positioned at different circumferential locations around the lid 110 and the container 102. For example, a first latch system 126A and a second latch system 126B may be positioned generally opposite one another on the lid 110 relative to the opening 106 of the container 102. In other examples, the humidifier 200 may include more than two latch systems distributed around the lid 110. The multiple latch systems 126 may be configured to apply a generally uniform clamping force around the perimeter of the lid 110 when engaged, thereby promoting consistent sealing between the lid 110 and the container 102.

The multi-latch configuration provides a non-threaded, non-rotational mechanism for securing the lid 110 relative to the container 102, such that the lid 110 is not required to be rotated relative to the container 102 during opening or closing. In other words, the lid 110 may be separated from and reattached to the container 102 through linear separation, without requiring rotational alignment or threading, which can reduce the likelihood of misalignment during reattachment. In addition, because the inlet port 116 and outlet port 118 are fixed to the lid 110, the lid 110 may be removed while maintaining a controlled orientation of tubing coupled to the inlet and outlet ports, thereby reducing the likelihood of tubing entanglement or twisting during refilling or maintenance. As used herein, linear separation refers to relative movement between the lid and container along a generally straight path without relative rotational motion between the components. Linear separation does not require movement along a particular axis or in a perfectly straight direction, and may include movement that is substantially linear while allowing minor angular deviation, tolerance-based motion, or incidental tilting during separation.

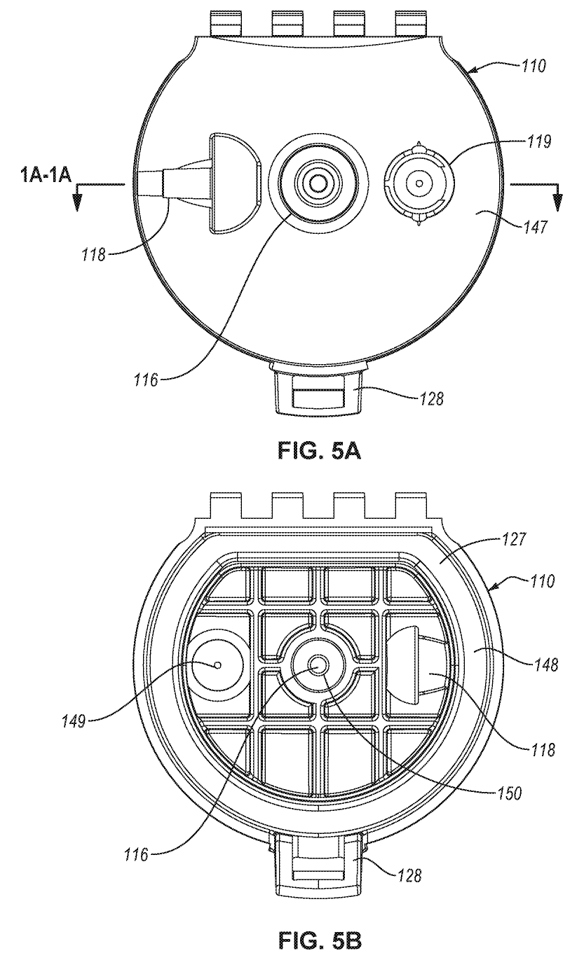

Referring to FIG. 5A, an exterior surface 147 of the lid 110 is shown. The lid 110 includes the inlet port 116, the outlet port 118, and a pressure relief valve 119. The inlet port 116, the outlet port 118, and the pressure relief valve 119 are fixed to the lid 110 such that they move together with the lid 110 during opening and closing of the humidifier 100. This arrangement allows inflow and outflow tubing coupled to the inlet port 116 and outlet port 118 to remain connected to the lid 110 during opening and closing, without requiring disconnection.

In some examples, the inlet port 116 includes internal threads 117 formed on an inner surface of the inlet port 116. The internal threads 117 are configured to engage a corresponding threaded adapter or connector of an inflow tube, adapter, or supply line, such as a connector associated with an oxygen concentrator or other gas supply device having a threaded outlet. Engagement of the threaded connector with the internal threads 117 provides a secure, releasable mechanical coupling between the inflow tube and the inlet port 116. In other examples, the inlet port 116 may be configured to receive a non-threaded connector, such as a push-fit, barbed, or friction-fit connector.

The inlet port 116 and outlet port 118 may be integrally formed with the lid 110 or formed as separate components that are sealably coupled to the lid 110. As shown, the inlet port 116 and the outlet port 118 extend from the exterior surface 147 of the lid 110 at different orientations relative to a central axis (C) of the container 102 when the lid 110 is in the closed position. However, the specific orientations and geometries of the inlet port 116 and outlet port 118 may vary depending on the configuration of the inflow and outflow tubing to be connected thereto. In some examples, the lid 110 includes visual indicia formed on the exterior surface 147 to identify the inlet port 116 and the outlet port 118, such as markings indicating fluid flow direction or port function (e.g., “IN” and “OUT”).

Referring to FIG. 5B, an interior surface 148 of the lid 110 includes internal portions of the inlet port 116, the outlet port 118, and the pressure relief valve 119. The inlet port 116 defines an internal channel 150 that extends from the exterior surface 147 of the lid 110 to the interior surface 148 and is configured to receive and fluidically couple to the fluid conduit 151 when the lid 110 is in the closed position. The fluid conduit 151 directs a gas from the inlet port 116 into the interior space 104 of the container 102, as described above. The outlet port 118 similarly defines an internal channel 149 that allows humidified gas to exit the interior space 104 and be conveyed to the outflow tube.

The interior surface 148 of the lid 110 further includes a recessed groove formed adjacent a circumferential edge of the lid 110, in which a gasket 127 is received. The gasket 127 is configured to sealingly engage the rim 107 of the opening 106 when the lid 110 is in the closed position. In some examples, the lid 110 includes opposing wall portions that define the recessed groove, such that the gasket 127 is positioned between the wall portions. When the lid 110 is closed, the rim 107 of the container 102 engages and compresses the gasket 127 within the recessed groove, thereby forming an airtight seal between the lid 110 and the container 102. The gasket 127 is configured to maintain an airtight seal between the lid 110 and the container 102 during normal operation of the humidifier 100, including at internal pressures below a predetermined pressure at which the pressure relief valve 119 is configured to activate. In this manner, the gasket 127 prevents unintended leakage of gas or liquid prior to activation of the pressure relief valve 119, while allowing the pressure relief valve 119 to perform controlled venting when the predetermined pressure is reached.

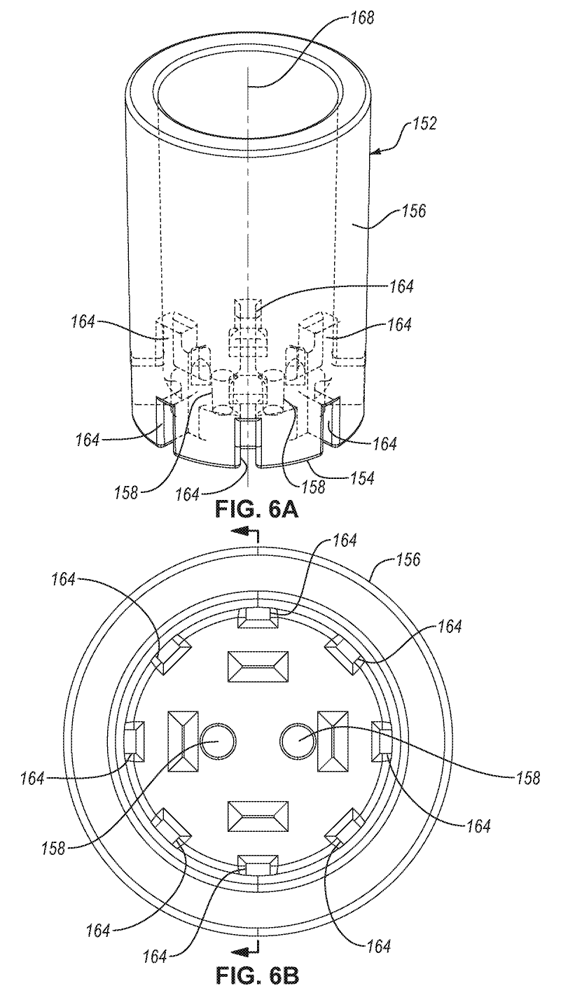

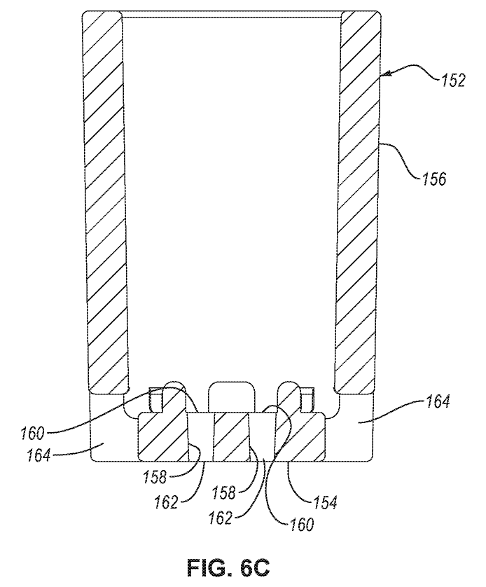

Referring to FIGS. 6A–6C, one example of the diffuser 152 is shown. As used herein, the term diffuser refers to a structure configured to disperse a gas into the interior space 104 of the container 102. As shown, the diffuser 152 is generally cylindrical and includes a sidewall 156 and a distal end 154. In other examples, the diffuser 152 may have different shapes or cross-sectional profiles. The diffuser 152 defines multiple passages that are fluidically coupled to the fluid conduit 151 and configured to expel gas into the liquid 109 within the container 102.

In some examples, the diffuser 152 includes a plurality of lateral passages 164 formed in the sidewall 156 of the diffuser 152. The lateral passages 164 are configured to expel a gas into the interior space 104 of the container 102 in directions substantially transverse to a longitudinal axis 168 of the diffuser 152. That is, each one of the lateral passages 164 includes an inlet opening and a corresponding outlet opening, connected by an internal channel that directs oxygen flow. The lateral passages 164 are distributed around the circumference of the diffuser 152 to promote dispersion of oxygen within the liquid 109. In some examples, the plurality of lateral passages 164 are distributed evenly around the circumference of the diffuser 152 for uniform oxygen dispersion from the lateral passages within the liquid. For example, the diffuser 152 may include eight (or more or less than eight) evenly spaced lateral passages arranged symmetrically around the sidewall 156. In other examples, the plurality of lateral passages 164 may be non-uniformly distributed around the circumference of the diffuser 152, such as concentrated in specific regions to target oxygen dispersion in certain areas of the liquid. The number, size, shape, and distribution of the lateral passages 164 may vary depending on the desired dispersion characteristics and flow conditions.

The diffuser 152 further includes at least one axial passage 158 formed in the distal end 154 of the diffuser 152. The axial passage 158 is oriented to expel a gas in a direction substantially parallel to the longitudinal axis 168 of the diffuser 152 when the lid 110 is in the closed position. In some examples, the axial passage 158 includes a tapered cross-section in a direction away from the lid 110, thereby producing a Venturi-like effect as a gas exits the diffuser 152. In other examples, the axial passage 158 maybe have a uniform cross-section.

Referring to FIG. 6C, a cross-sectional view through two axial passages 158 shows an example of a tapered geometry of the axial passages 158. The tapered geometry increases the velocity of a gas as it exits the diffuser 152, which helps to enhance the dispersion and mixing of the gas within the liquid in the container 102. In one example, the axial passage 158 extends through the distal end 154 of the diffuser 152 and is defined by a first end 160 and a second end 162, where the first end 160 has a larger cross-sectional area than the second end 162. The transition between the first end 160 and the second end 162 may include a tapered or conical profile. In some examples, the taper extends along a length of the axial passage 158 at an angle of between, and inclusive of, 2 and 10 degrees, in other examples, between, and inclusive of, 2 and 5 degrees, and in yet other examples, the taper is approximately 4 degrees.

The Venturi-like effect generated by the axial passage 158 allows the humidifier 100 to operate effectively across a wide range of flow rates, making it suitable for diverse applications. For example, the diffuser 152 may accommodate flow rates ranging from about 2 liters-per-minute (l/min) to 15 l/min. In other words, the diffuser 152 has flexibility for use in both low and high flow conditions. At lower flow rates, such as below 4 l/min, the Venturi-like effect facilitates thorough oxygen dispersion within the liquid. At higher flow rates, the momentum provided by the axial passage 158 provides adequate oxygen mixing and uniform aeration. In some cases, the diffuser 152 can achieve a target humidity level of approximately 40%, at low flow rates (e.g., 2 l/min) and high flow rates.

The number, size, and shape of the axial passages 158 can vary. In one example, the diffuser 152 includes two axial passages 158, spaced apart on the distal end 154 of the diffuser 152. In other examples, the diffuser 152 may include more than two axial passages 158, symmetrically arranged on the distal end 154 to provide balanced oxygen distribution. In yet other examples, the diffuser 152 may include a single axial passage 158, positioned along the longitudinal axis 168 of the diffuser 152. Each one of the axial passages 158 may have a smooth inner surface to reduce flow resistance and maintain consistent oxygen delivery. Alternatively, the axial passage 158 may include structural features, such as ridges or grooves, on the inner surface, to further enhance turbulence and promote mixing of the oxygen within the liquid in the container 102. The size of the axial passages 158 are determined according to a desired bubble size.

Referring to FIGS. 7A and 7B, one example of the pressure relief valve 119 is shown. The pressure relief valve 119 is coupled to the lid 110 and includes a housing 170 that defines an interior channel 172. The housing 170 includes at least one exhaust port 174 in fluid communication with the interior channel 172, through which pressurized gas from within the container 102 may be vented from the container 102.

A valve member 180 is positioned within the interior channel 172 and is movable between, and inclusive of, a closed position, as shown in FIG. 7A, and an open position, as shown in FIG. 7B. In the closed position, the valve member 180 covers the at least one exhaust port 174 to inhibit venting of gas through the pressure relief valve 119. In the open position, the valve member 180 is displaced relative to the exhaust port 174 such that the exhaust port 174 is uncovered, enabling pressurized gas to be vented from the interior channel 172. The valve member 180 is configured to move axially along a longitudinal axis 175 between the closed position and the open position in response to pressure conditions within the container 102. In some examples, the valve member 180 has a weight selected to bias the valve member 180 toward the closed position under normal operating conditions and to provide a sealing engagement with the exhaust port 174, while enabling movement to the open position when a predetermined internal pressure is exceeded.

In some examples, the housing 170 includes a transparent portion 176 positioned at the top end of the housing 170, which enables a user to visually observe the position of the valve member 180 during operation of the pressure relief valve 119. That is, the transparent portion 176 provides visual access to at least a portion of the interior channel 172. For example, the transparent portion 176 may be a clear cap or window that allows the user to see whether the valve member 180 has moved from the closed position to the open position. When the valve member 180 is in the open position, the transparent portion 176 enables visual observation of the valve member 180, thereby providing a visual indication that the pressure relief valve 119 has been activated. In other examples, the housing 170 does not include a transparent portion 176.

Additionally, or alternatively, in some examples, the pressure relief valve 119 provides an audible indication when the valve member 180 moves to the open position. For example, the housing 170 may include a whistle vent 181 formed in a sidewall of the housing 170. When gas flows through the pressure relief valve 119 in the open position, interaction between the gas flow and the whistle vent 181 may produce an audible sound indicating excessive pressure or flow restriction.

Movement of the valve member 180 between the closed position and the open position occurs in response to pressure conditions within the container 102. When internal pressure exceeds a predetermined threshold, the valve member 180 moves to the open position to enable venting of gas. In some examples, the predetermined threshold is in the range of between about 2 and 12 psi. In other examples, the predetermined threshold is in the range of between about 3 and 8 psi. In yet other examples, the predetermined threshold is approximately 6 psi. When internal pressure decreases below the predetermined threshold, the valve member 180 returns to the closed position to inhibit further venting. When the valve member 180 is in the open position, the transparent portion 176 of the housing 170 enables visual observation of the valve member 180, thereby providing a visual indication that the pressure relief valve 119 has been activated.

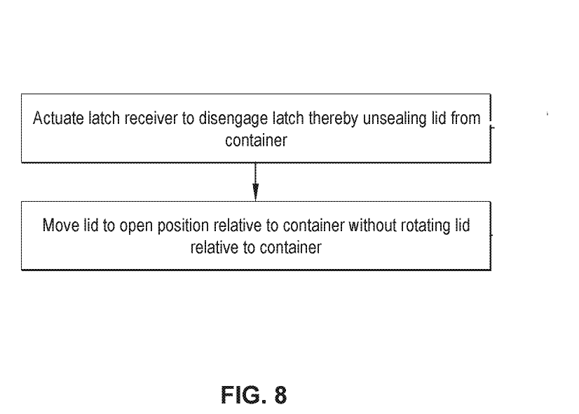

Referring to FIG. 8, one example method 300 of accessing the interior space 104 of the humidifier 100 is illustrated. At block 302, the method 300 includes actuating a latch receiver 136 of at least one latch system 126 to disengage a corresponding latch 128 from the latch receiver 136, thereby unsealing the lid 110 from the container 102. The latch receiver 136 may be actuated by a user to move the locking projection 140 out of engagement with the latch opening 130 of the latch 128.

At block 304, after disengagement of the latch system 126, the method 300 includes moving the lid 110 relative to the container 102 from a closed position to an open position without rotating the lid 110 relative to the container 102. In some examples, the lid 110 is pivoted about a hinge while remaining attached to the container 102. In other examples, the lid 110 is removed from the container 102 by linear separation following release of multiple latch systems. In both cases, the lid 110 may be opened or removed while maintaining tubing coupled to the inlet port 116 and the outlet port 118 in a controlled orientation, thereby reducing the likelihood of tubing entanglement, twisting, or disconnection during access to the interior space 104.

In some examples of the method 300, moving the lid 110 relative to the container 102 includes pivoting the lid 110 about a hinge 112 while the lid 110 remains attached to the container 102. In such examples, after actuation of the latch receiver 136 to disengage the latch system 126, the lid 110 pivots about the rotation axis defined by the hinge 112 from the closed position to the open position. The hinged movement allows the lid 110 to remain permanently coupled to the container 102 during opening and closing, thereby reducing the likelihood of misalignment during reclosure and enabling inlet and outlet tubing coupled to the lid 110 to remain connected throughout the opening process.

In other examples of the method 300, moving the lid 110 relative to the container 102 includes separating the lid 110 from the container 102 after disengaging a multiple latch systems 126. In such examples, actuation of each latch receiver 136 disengages the corresponding latch 128, permitting removal of the lid 110 from the container 102 without relative rotation between the lid 110 and the container 102. The lid 110 may be removed by linear separation, allowing the lid 110 to be lifted away from the container 102 while maintaining a controlled orientation of tubing coupled to the inlet port 116 and the outlet port 118.

In some examples, the method 300 further includes reattaching the lid 110 to the container 102 by moving the lid 110 from the open position to the closed position without rotating the lid 110 relative to the container 102. As the lid 110 is returned to the closed position, the latch 128 and latch receiver 136 of the at least one latch system 126 re-engage to secure the lid 110 relative to the container 102. In some examples, engagement of the latch system 126 produces an audible indication, such as a clicking sound, that confirms proper engagement of the latch 128 with the latch receiver 136. This audible indication may provide user feedback that the lid 110 is fully secured in the closed position prior to operation of the humidifier 100.

The oxygen humidifier systems and methods disclosed herein provide improved mechanisms for accessing an interior space of a humidifier while maintaining controlled orientation of connected tubing and reducing the likelihood of misalignment, leakage, or user error. The disclosed configurations enable a lid to be opened or removed without relative rotation between the lid and the container, including hinged and multi-latch arrangements that support non-rotational separation and reattachment. Additional features, such as latch systems with user feedback, protective rib structures, planar container surfaces, non-rotational sealing interfaces, and a pressure relief valve with visual indication, contribute to enhanced usability, safety, and reliability during operation and maintenance. Collectively, these features address shortcomings of conventional threaded or rotational humidifier designs while providing a modular, user-friendly architecture adaptable to a range of respiratory gas delivery applications.

In the above description, certain terms may be used such as "up," "down," "upper," "lower," "horizontal," "vertical," "left," "right," “over,” “under” and the like. These terms are used, where applicable, to provide some clarity of description when dealing with relative relationships. But, these terms are not intended to imply absolute relationships, positions, and/or orientations. For example, with respect to an object, an "upper" surface can become a "lower" surface simply by turning the object over. Nevertheless, it is still the same object. Further, the terms “including,” “comprising,” “having,” and variations thereof mean “including but not limited to” unless expressly specified otherwise. An enumerated listing of items does not imply that any or all of the items are mutually exclusive and/or mutually inclusive, unless expressly specified otherwise. The terms “a,” “an,” and “the” also refer to “one or more” unless expressly specified otherwise. Further, the term “plurality” can be defined as “at least two.” Moreover, unless otherwise noted, as defined herein a plurality of particular features does not necessarily mean every particular feature of an entire set or class of the particular features.

The term “about” or “substantially” in some embodiments, is defined to mean within +/-5% of a given value, however in additional embodiments any disclosure of “about” may be further narrowed and claimed to mean within +/- 4% of a given value, within +/- 3% of a given value, within +/- 2% of a given value, within +/- 1% of a given value, or the exact given value. Further, when at least two values of a variable are disclosed, such disclosure is specifically intended to include the range between the two values regardless of whether they are disclosed with respect to separate embodiments or examples, and specifically intended to include the range of at least the smaller of the two values and/or no more than the larger of the two values. Additionally, when at least three values of a variable are disclosed, such disclosure is specifically intended to include the range between any two of the values regardless of whether they are disclosed with respect to separate embodiments or examples, and specifically intended to include the range of at least the A value and/or no more than the B value, where A may be any of the disclosed values other than the largest disclosed value, and B may be any of the disclosed values other than the smallest disclosed value.

Additionally, instances in this specification where one element is “coupled” to another element can include direct and indirect coupling. Direct coupling can be defined as one element coupled to and in some contact with another element. Indirect coupling can be defined as coupling between two elements not in direct contact with each other, but having one or more additional elements between the coupled elements. Further, as used herein, securing one element to another element can include direct securing and indirect securing. Additionally, as used herein, “adjacent” does not necessarily denote contact. For example, one element can be adjacent another element without being in contact with that element.

As used herein, the phrase “at least one of”, when used with a list of items, means different combinations of one or more of the listed items may be used and only one of the items in the list may be needed. The item may be a particular object, thing, or category. In other words, “at least one of” means any combination of items or number of items may be used from the list, but not all of the items in the list may be required. For example, “at least one of item A, item B, and item C” may mean item A; item A and item B; item B; item A, item B, and item C; or item B and item C. In some cases, “at least one of item A, item B, and item C” may mean, for example, without limitation, two of item A, one of item B, and ten of item C; four of item B and seven of item C; or some other suitable combination.

Unless otherwise indicated, the terms "first," "second," etc. are used herein merely as labels, and are not intended to impose ordinal, positional, or hierarchical requirements on the items to which these terms refer. Moreover, reference to, e.g., a “second” item does not require or preclude the existence of, e.g., a “first” or lower-numbered item, and/or, e.g., a “third” or higher-numbered item.

As used herein, a system, apparatus, structure, article, element, component, or hardware “configured to” perform a specified function is indeed capable of performing the specified function without any alteration, rather than merely having potential to perform the specified function after further modification. In other words, the system, apparatus, structure, article, element, component, or hardware “configured to” perform a specified function is specifically selected, created, implemented, utilized, programmed, and/or designed for the purpose of performing the specified function. As used herein, “configured to” denotes existing characteristics of a system, apparatus, structure, article, element, component, or hardware which enable the system, apparatus, structure, article, element, component, or hardware to perform the specified function without further modification. For purposes of this disclosure, a system, apparatus, structure, article, element, component, or hardware described as being “configured to” perform a particular function may additionally or alternatively be described as being “adapted to” and/or as being “operative to” perform that function.

The schematic flow chart diagrams included herein are generally set forth as logical flow chart diagrams. As such, the depicted order and labeled steps are indicative of one example of the presented method. Other steps and methods may be conceived that are equivalent in function, logic, or effect to one or more steps, or portions thereof, of the illustrated method. Additionally, the format and symbols employed are provided to explain the logical steps of the method and are understood not to limit the scope of the method. Although various arrow types and line types may be employed in the flow chart diagrams, they are understood not to limit the scope of the corresponding method. Indeed, some arrows or other connectors may be used to indicate only the logical flow of the method. For instance, an arrow may indicate a waiting or monitoring period of unspecified duration between enumerated steps of the depicted method. Additionally, the order in which a particular method occurs may or may not strictly adhere to the order of the corresponding steps shown.

The present subject matter may be embodied in other specific forms without departing from its spirit or essential characteristics. The described examples are to be considered in all respects only as illustrative and not restrictive. All changes which come within the meaning and range of equivalency of the claims are to be embraced within their scope.

What is claimed is:

1. An oxygen humidifier comprising a container defining an interior space and an opening for accessing the interior space; a lid pivotably coupled to the container via a hinge that defines a rotation axis about which the lid pivots relative to the container; and a latch system comprising a latch coupled to the lid and a latch receiver coupled to the container, the latch and latch receiver being configured to releasably secure the lid to the container; wherein: the lid comprises an inlet port and an outlet port fixed to the lid; the lid is selectively pivotable about the rotation axis between, and inclusive of, an open position, in which the lid is unsealed against the container to enable access to the interior space through the opening of the container, and a closed position, in which the lid forms a seal with the container; and when the lid pivots from the closed position to the open position, the lid pivots away from and remains outside of the interior space.

2. The oxygen humidifier of claim 1, wherein: the latch of the latch system defines a latch opening; the latch receiver of the latch system comprises a release button and a locking projection operatively coupled to the release button; the locking projection is configured to engage the latch opening to retain the lid in the closed position; and actuation of the release button disengages the locking projection from the latch opening.

3. The oxygen humidifier of claim 1, wherein the latch system is positioned on the lid at a location opposite the hinge relative to the opening of the container.

4. The oxygen humidifier of claim 1, further comprising a diffuser fluidically coupled to the inlet port and positioned within the interior space of the container when the lid is in the closed position, wherein the diffuser comprises: a plurality of lateral passages formed in a sidewall of the diffuser and oriented to expel a fluid in a direction substantially normal to a longitudinal axis of the diffuser; and at least one axial passage formed in a distal end of the diffuser and oriented to expel the fluid in a direction substantially parallel to the longitudinal axis of the diffuser.

5. The oxygen humidifier of claim 4, wherein the at least one axial passage has a tapered cross-section in a direction away from the lid.

6. The oxygen humidifier of claim 1, further comprising a pressure relief valve coupled to the lid and comprising: a housing defining an interior channel and at least one exhaust port in fluid communication with the interior channel, the housing comprising a transparent portion; and a valve member positioned within the interior channel and movable between, and inclusive of, a closed position, in which the valve member covers the at least one exhaust port, and an open position, in which the valve member uncovers the at least one exhaust port; wherein the transparent portion enables observation of the valve member when the pressure relief valve is in the open position.

7. The oxygen humidifier of claim 1, further comprising a gasket coupled within a recessed groove of an interior surface of the lid, wherein: the gasket is configured to sealingly engage a rim of the container, that defines the opening when the lid is in the closed position to form a seal between the lid and the container; and the recessed groove is defined between opposing wall portions of the interior surface that retain the gasket.

8. The oxygen humidifier of claim 1, wherein the container has at least one planar portion on an exterior surface of the container.

9. The oxygen humidifier of claim 1, further comprising one or more protective ribs formed on the container adjacent to the latch receiver, wherein the one or more protective ribs are positioned to shield the latch receiver from inadvertent actuation.

10. The oxygen humidifier of claim 1, wherein the inlet port includes internal threads formed on an inner surface of the inlet port, the internal threads being configured to engage a corresponding threaded connector of an inflow tube.

11. The oxygen humidifier of claim 1, wherein engagement between the latch and the latch receiver provides an audible indication that the lid is secured in the closed position.

12. An oxygen humidifier comprising: a container defining an interior space and an opening for accessing the interior space;
a lid removably coupled to the container; and multiple latch systems, each one of the multiple latch systems comprising a latch coupled to the lid and a latch receiver coupled to the container, the latch and latch receiver of each one of the multiple latch systems being configured to releasably secure the lid to the container; wherein: the lid comprises an inlet port and an outlet port fixed to the lid; the lid is selectively movable between an open position, in which the lid is unsealed against the container to enable access to the interior space through the opening of the container, and a closed position, in which the lid forms a seal with the container; and when the lid is in the closed position, the latch and the latch receiver of each one of the multiple latch systems are engaged to retain the lid in sealed engagement with the container; and when the lid is moved from the closed position to the open position, actuation of the latch receiver of each one of the multiple latch systems disengages the corresponding latch to enable separation of the lid from the container without rotation of the lid relative to the container about a central axis of the container.

13. The oxygen humidifier of claim 12, wherein: the multiple latch systems comprises two latch systems, the two latch systems including a first latch system and a second latch system; and the first latch system is positioned opposite of the second latch system on the lid relative to the opening of the container.

14. The oxygen humidifier of claim 12, wherein the latch of each one of the multiple latch systems defines a latch opening; the latch receiver of each one of the multiple latch systems comprises a release button and a locking projection operatively coupled to the release button; the locking projection is configured to engage the latch opening to retain the lid in the closed position; and actuation of the release button disengages the locking projection from the latch opening.

15. The oxygen humidifier of claim 12, further comprising one or more protective ribs formed on the container adjacent to the latch receiver of each one of the multiple latch systems, wherein the one or more protective ribs are positioned to shield the latch receiver from inadvertent actuation.

16. The oxygen humidifier of claim 12, wherein engagement between the latch and the latch receiver of each one of the multiple latch systems provides an audible indication that the corresponding one of the multiple latch systems is secured.

17. A method of accessing an interior space of an oxygen humidifier, the method comprising: actuating a latch receiver of at least one latch system to disengage a latch from the latch receiver, the latch receiver being coupled to a container of the oxygen humidifier and the latch being coupled to a lid, thereby unsealing the lid from the container; and moving the lid relative to the container from a closed position to an open position without rotating the lid relative to the container about a central axis of the container, while maintaining fluid tubing connected to an inlet port and an outlet port of the lid.

18. The method of claim 17, wherein moving the lid relative to the container comprises pivoting the lid about a hinge while the lid remains attached to the container.

19. The method of claim 17, wherein moving the lid relative to the container comprises separating the lid from the container by linear separation after disengaging a multiple latch systems.

20. The method of claim 17, further comprising: moving the lid relative to the container from the open position to the closed position without rotating the lid relative to the container; and receiving an audible indication upon engagement of the latch and the latch receiver of the at least one latch system when the lid is in the closed position.
